(19) United States
(12) Patent Application Publication
(10) Pub. No.: US 2003/0153009 A1
(43) Pub. Date: Aug. 14, 2003

(54) Method for influencing kinase activity with AG879

(76) Inventors: Hong He, Parkville/Melbourne (AU); Hiroshi Maruta, Parkville/Melbourne (AU)

Correspondence Address: FULBRIGHT & JAWORSKI, LLP, 666 FIFTH AVE, NEW YORK, NY, 10103-3198, US

(21) Appl. No.: 10/074,871

(22) Filed: Feb. 12, 2002

(51) Int. Cl. G01N 33/573

(52) U.S. Cl. 435/7.4

(57) ABSTRACT

The invention relates to the use of AG879 and its derivatives as kinase inhibitors. The molecules can be used, per se, as inhibitors, or they can be used in connection with screening assays to identify modifiers of kinase activity.

### FIELD OF THE INVENTION

This invention relates to methods for identifying molecules which modulate tyrosine kinase pathways, especially those involving the molecules known as PAK1, FAK and ETK.

### BACKGROUND AND PRIOR ART

“PAK1” is a member of the CDC42/Rac-dependent, Ser/Thr kinase family of “PAKs.” It is activated by oncogenic RAS mutants, such as v-Ha-RAS, and has been shown to be essential for RAS transformation of fibroblasts, such as Rat-1, and NIH 3T3 cells. See Tang, et. al., Mol. Cell Biol. 17:4454-4464 (1997); He, et. al, Cancer J. 7:191-202 (2001), both of which are incorporated by reference. He, et. al., have elucidated several distinct pathways as being essential for v-Ha-RAS induced activation in these cells. One such pathway involves PI-3 kinase, which produces phosphatidyl-inositol 3,4,5 triphosphate, or “PIP,” which activates both CDC42 and Rac GTPase, through a GDP dissociation stimulator (“GDS”), referred to as VAV.

A second pathway involves “PIX,” which is an SH3 protein that binds a Pro rich domain, referred to as “PAK18,” that is located in between the N-terminal, GTPase binding domain, and the C-terminal kinase domain of PAK1. See Manser, et. al, Mol. Cell 1:183-192 (1998). PIX has been shown to bind the protein referred to as “CAT,” which is known to be a substrate for Src family kinases. See Bagrodia, et. al., J. Biol Chem 274:22393-22400 (1999).

Yet a third pathway involves the protein referred to as “NCK,” which is an SH2/SH3 adaptor protein. See Galisteo, et. al., J. Biol. Chem 271:20997-21000 (1996). The SH3 domain of NCK binds another Pro rich domain of PAK1, located near the N terminus, while the SH2 domain binds the tyr phosphorylated EGF receptor referred to as ErbB1. When ErbB1 is activated by EGF, PAK1 is translocated to the plasma membrane via NCK. The involvement of both Src family kinases, and ErBB1 in PAK1 activation is supported by prior findings that both the known Src family kinase inhibitor “PP1,” and “AG1478”, which is a known, specific ErbB1 inhibitor, block RAS induced PAK1 activation, and transformation, both in vitro and in vivo. See He, et. al., supra; He, et. al, Cancer J. 6:243-248 (2000), incorporated by reference. Yet a fourth pathway involves ErbB2, a member of the ErbB family of Tyr kinases. See He, et. al., Cancer J. 7:191-202 (2001).

Many of the small molecule tyrosine kinase inhibitors can be found in Levitzki, et. al., “Tyrosine Kinase Inhibition: An Approach To Drug Development,” Science 267:1782-1788 (1995), incorporated by reference. This reference discloses, inter alia “AG1478,” discussed supra, as well as AG825, which has been shown by He, et. al., supra, to block RAS induced activation of PAK1, and malignant transformation of cells. The “IC₅₀” for AG825 in this context is described as about 0.35 μM.

Yet another pathway involves the beta integrin, FAK, and ETK molecules. Beta integrin activates the Tyr kinase “FAK,” which in turn phosphorylates and activates the ETK molecule. See Chen, et. al., Nat Cell Biol. 3:439-444 (2001), incorporated by reference. ETK is itself a member of the TEC/BTK family of Tyr kinases. See, e.g., Qiu, et. al., Oncogene 19:5651-5661 (2001); Smith, et. al., Bioassays 23:436-446 (2001), both of which are incorporated by reference. ETK carries an N-terminal, pleckstrin homology domain (“PH” hereafter), which is followed immediately by a TEC homology domain. See Qiu, et. al., supra; Smith, et. al., supra. All of the structural and functional information notwithstanding, what is unknown is whether RAS activation requires the integrin/FAK/ETK pathway described herein, and how RAS activates this pathway.

Previous experiments showed that AG879, described by Levitzki, et. al, supra was an inhibitor for ErbB2 and FLK-1, and suppressed the growth of RAS induced sarcomas in vivo, using a nude mouse model.

In view of these results, studies were carried out to attempt to elicit mechanisms involving PAK1. It was found that AG879 inhibits both RAS induced activation, and Tyr phosphorylation of PAK1, by blocking ETK. These observations will be elaborated upon more fully in the disclosure which follows.

### BRIEF DESCRIPTION OF THE FIGURES

FIG. 1 sets forth the structures of the molecules referred to herein, e.g., “AG879” et. al. These are based on Levitzki, et. al., supra.

FIG. 2 presents, graphically, results of studies carried out to determine the effect of AG879 on PAK1.

FIG. 3 shows the effect of AG879 on anchorage dependent growth of transformed RAS cells.

FIG. 4 depicts results of experiments designed to test the effect of AG879 on tyrosine phosphorylation of ETK.

FIG. 5 shows results of experiments designed to show if AG879 inhibited tyr phosphorylation of PAK1.

FIG. 6 shows that AG879 inhibited kinase activity of ETK.

FIG. 7 shows AG879 suppressed Tyr phosphorylation of FAK.

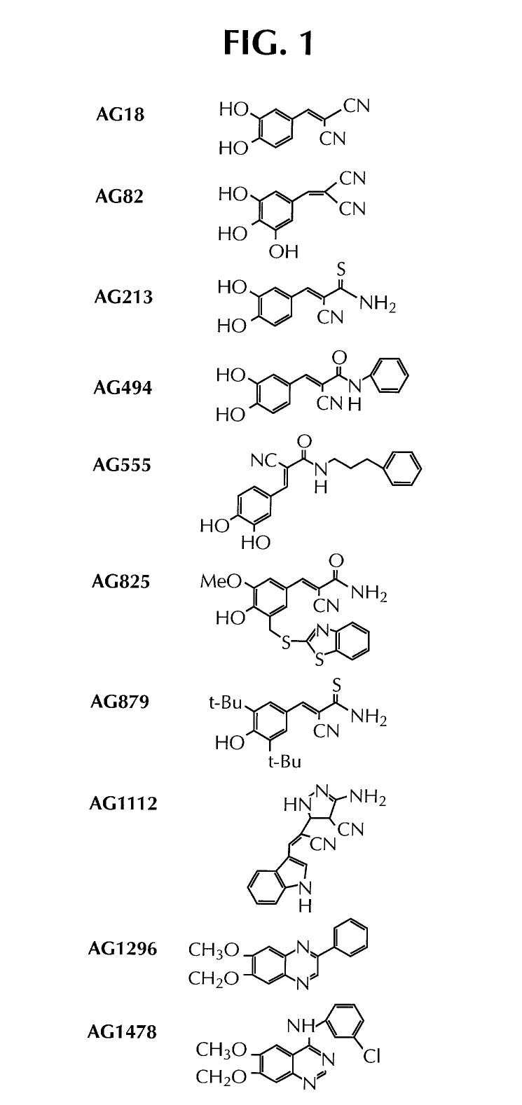

### DETAILED DESCRIPTION OF PREFERRED EMBODIMENTS

### EXAMPLE 1

This example describes experiments designed to determine the effect of AG879 on PAK1.

RAS cells, which are NIH 3T3 fibroblasts transformed with v-Ha-RAS, were serum starved, overnight, and were then incubated with varying concentrations of AG879 (0.01-10 μM), for 1 hour. Following the hour of culture, the cells were lysed in lysis buffer (40 mM HEPES, pH 7.4, 1% Nonidet P-40, 1 mM EDTA, 100 mM NaCl, 25 mM NaF, 100 μM NaVO₃, 1 MM phenylmethyl sulfonyl fluoride, and 100 units/ml aprotonin). Lysates were tested, via Bradford assay, to determine protein content, and those containing 1 mg of protein were admixed with an anti-PAK1 antibody to immunoprecipitate proteins.

Once the proteins were immunoprecipitated, they were analyzed in a PAK kinase assay, as described by Tang, et. al., Mol. Cell Biol 17: 4454-4464 (1997); He, et. al., Cancer J. 7:191-202 (2001), He, et. al., Cancer J 6:243-248 (2000); Obemeier, et. al., EMBO J 17:4328-4339 (1998), all of which are incorporated by reference.

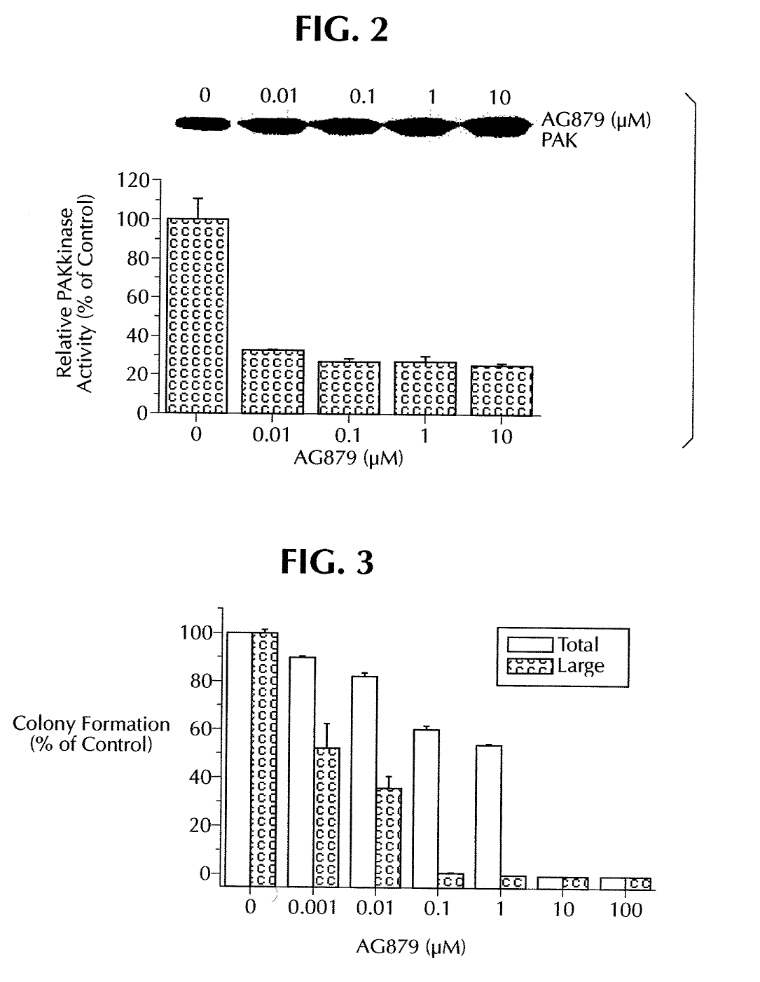

FIG. 2 shows these results. Essentially, at all concentrations tested, the protein content of the precipitates did not vary; however, kinase activity was blocked quite strongly.

In follow up experiments a fusion protein “GST-PAK1” was prepared, and tested in the same way. The AG879 did not inhibit the kinase properties of the fusion protein, even at 10 μM concentrations. The result from these two experiments suggest that ErbB2 is not involved in the inactivation of PAK1 by AG879.

### EXAMPLE 2

These experiments were designed to determine the effect of AG879 on anchorage dependent growth of RAS cells. To do this, 10³ cells, per plate, were seeded into 0.35% top agar, where the medium contained different concentrations of AG879, ranging from 1 nM to 100 μM. The cultures were then incubated for 3 weeks, in accordance with He, et. al., Cancer J. 7:191-202 (2001); He, et. al., Cancer J 6:243-248 (2000); Maruta, et. al., J. Biol Chem 266:11661-11668 (1991), all of which are incorporated by reference. After three weeks, the colonies were stained, and counted, using standard methods. The control used was non-treated cells, plated into the same type of medium.

Results, which are shown in FIG. 3, are averages of two experiments. In these results, “large” represents the number of colonies containing more than 100 cells, while “total” includes all colonies.

The result of these experiments indicate that the IC₅₀ for large colony formation is around 10 nM. This is shown, graphically, in FIG. 3. The results show that AG879 suppresses anchorage independent growth of RAS transformants.

In follow up experiments, increasing the concentration of AG879 to as much as 1 μM did not effect anchorage dependent growth. These results, in tandem, i.e., the suppression of PAK1 activation, and suppression of RAS induced malignant transformation, suggest that AG879's activity is not linked to blocking ErbB2, but via some other, PAK1 associated kinase. The experiments which follow were designed to address this question.

### EXAMPLE 3

M^{c}Manus, et. al., J. Biol. Chem 275:35328-35334 (2000), incorporated by reference, showed that tyr phosphorylation of PAK1 is required for its Ser/Thr kinase activity. They showed this by treating PAK1 with tyr phosphatase when its activity was reduced. Bagheri-Yarmand, et. al., J. Biol. Chem. 276:24903-29404 (2001), incorporated by reference, showed that the “ETK” enzyme associates with PAK1 through its PH domain, and activates PAK1 via phosphorylation. Experiments were designed to determine if AG879 affected the Tyr activity of ETK. To do so, serum starved RAS cells were treated, with varying concentrations of AG879 for 1 hour. Cell lysates were prepared using, as lysis buffer, 20 mM Tris-HCl (pH 7.5), 100 mM NaCl, 10% glycerol, 1% Nonidet P-40, 10 mM NaF, 100 μM NaVO₃, 1 mM phenylmethylsulfonyl fluoride, and 100 units/ml of aprotonin. Lysates were treated either with a rabbit, anti-ETK antibody, as an anti-phospo Tyr antibody, to immunoprecipitate the enzyme, and an ETK kinase assay was performed, in accordance with Chen, et. al., Nat. Cell Biol. 3:439-444 (2001); and Bagheri-Yarmand, et. al., supra, both of which are incorporated by reference. Endogenous PAK1, associated with ETK was used as a substrate, in either the absence, or varying concentrations of AG879.

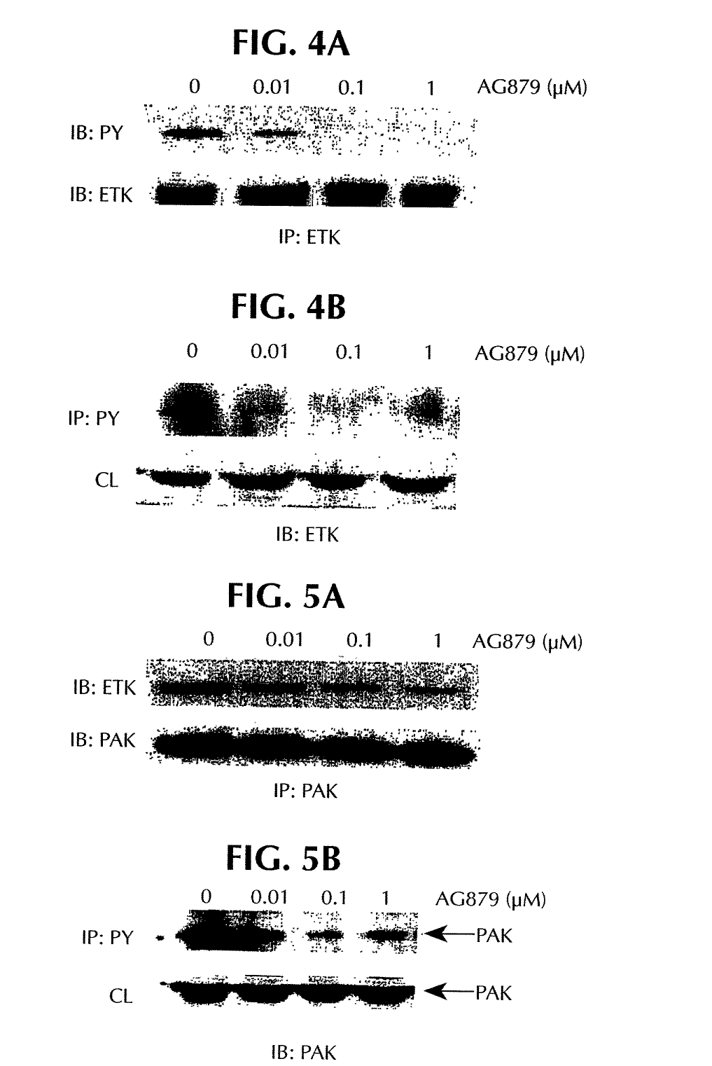

The results are presented in FIGS. 4 and 5. FIG. 4 shows that the compound inhibited tyrosine phosphorylation of ETK at 10 nM, but did not affect the level of the protein. In addition, the compound was shown to suppress ETK-PAK1 association, and also reduced Tyr phosphorylation of PAK1, at 10 nM concentrations. This is shown in FIG. 5.

The results as depicted in FIGS. 4 and 5 show that AG879 does inhibit the kinase activity of ETK, blocking its auto-phosphorylation, its association with PAK1, and its ability to phosphorylate PAK1.

### EXAMPLE 4

These experiments were designed to determined how AG879 inhibited ETK activity, i.e., was the inhibition direct, or indirect? To test this, RAS cells were again lysed and immunoprecipitated with an anti-ETK antibody, as described supra. Following this, the immunoprecipitates were used in an in vitro kinase assay, using no, or varying concentrations, of AG879 (0.001-10 μM), as described supra. Two, independent experiments, were carried out.

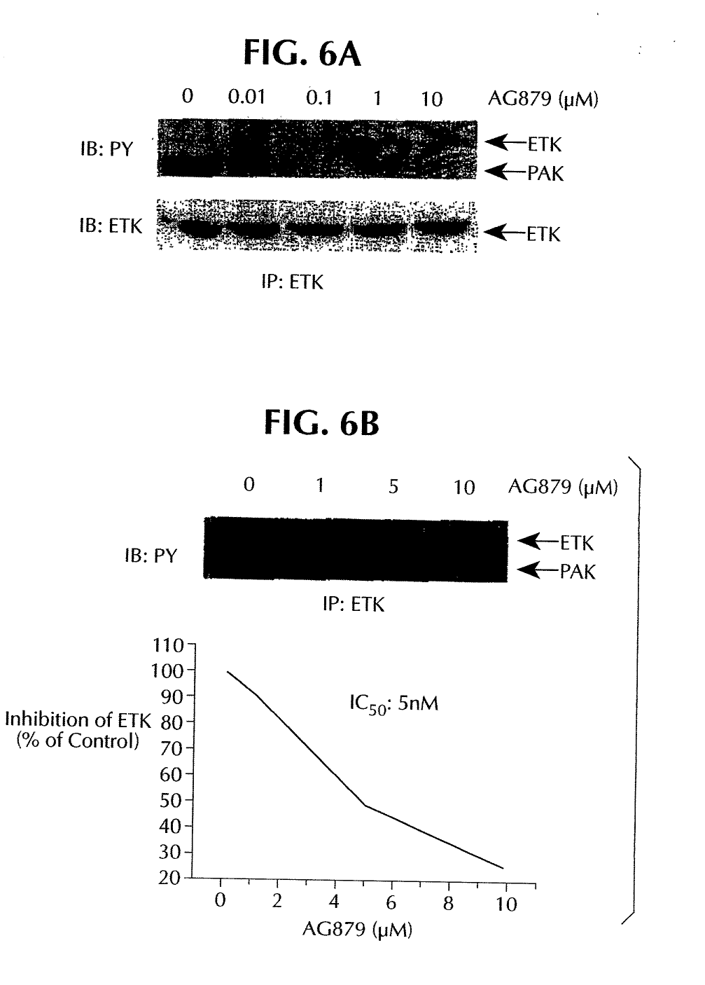

The results indicated that at 10 nM, AG879 inhibited the Tyr phosphorylation of PAK1 by ETK, and also Tyr phosphorylation of ETK. The IC₅₀ was about 5 nM. FIG. 6 shows this.

In additional experiments which paralleled these, but used other members of the TEC family (kinases such as TEC, BTK, and ITK), the molecule had no inhibitory effect, even when the concentration employed was as high as 10 μM. These results show, clearly, that ETK is a direct target of AG879.

### EXAMPLE 5

Qiu, et. al., Oncogene 19: 5651-5661 (2000), and Smith et. al., Bioassays 23: 436-446 (2001), both of which are incorporated by reference, describe ETK as a cytoplasmic, or non-receptor, tyrosine kinase, activated at the plasma membrane. It has been shown, recently, by Chen, et. al., Nat. Cell Biol. 3: 439-444 (2001), that the activation of ETK by extracellular matrix is regulated by the molecule “FAK.” through interaction between the PH domain of ETK and the FERM domain of FAK. Chen, et. al., also show that activated FAK binds ETK and elevates Tyr phosphorylation of ETK.

Chishti, et. al., Trends Biochem. Sci. 23: 281-282 (1998), and Tsai, et. al., Mol. Cell Biol. 20: 2043-2054 (2000), have shown that the N-terminus of FAK shares significant sequence homology with FERM domains.

This information suggested experiments to determine if AG879 affected the FAK-ETK interaction. In order to determine this, RAS cells were again serum starved, lysed, and immunoprecipitated using an anti-FAK antibody. The precipitates were then blotted, using either anti-phospo Tyr, anti-ETK, or anti-PAK1 antibodies, separately.

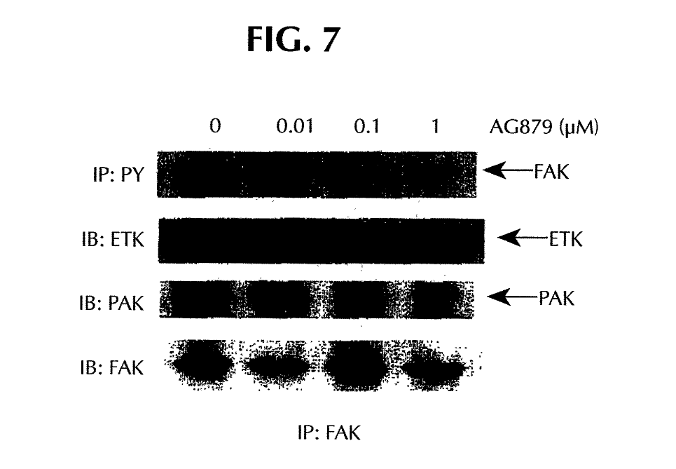

The results, presented in FIG. 7, showed that AG879 suppressed Tyr phosphorylation of FAK and its association with ETK at 100 nM, but not at the lower concentration of 10 nM. The molecule did in fact inhibit FAK-PAK1 interaction at the lower concentration of 10 nM. These results suggest that PAK1 associates with FAK via ETK, and also suggest that once a PAK1-ETK complex is disrupted by AG879, PAK1 can no longer interact with FAK.

The foregoing disclosure sets forth various features of the invention, which include assays for identifying agents which modulate the kinase associated pathways described herein. It has shown, for example, that AG879 (i) inhibits the kinase activity of PAK1, such as PAK1 in immuno precipitated form, (ii) prevented colony formation of RAS transformed cells, (iii) inhibited the activity of ETK, including its ability to autophosphorylate, its ability to associate with PAK1, and its ability to phosphorylate PAK1, and (iv) blocked the phosphorylation of FAK and its association with ETK. Hence, given that one can establish a “standard” with AG879, in any of the assays described supra, one can test a compound, or formulation of interest in any of these assays, and compare the results thus secured with results obtained using AG879. By comparing these values, one can determine whether a test compound or formulation modulates a PAK1 associated pathway, such as by agonizing involved molecules or antagonizing these. Any type of molecule, including “small molecules,” such as AG879 or other naturally occurring molecules such as those described by Levitzki, et al, supra, proteins, including peptides, antibodies, antibody fragments, and so forth, portions of kinase molecules, and other proteins can be tested for their ability to modulate the PAK1 associated pathways described herein. Similarly, molecules such as lipids, carbohydrates, molecules containing lipid or carbohydrate moieties, etc., can also be tested.

The assays of the invention may be carried out in vitro or in vivo, using complete enzyme molecules, or portions of, e.g., PAK1, FAK, ETK or other molecules involved in the relevant pathways described herein.

A polypeptide or peptide as described herein can be used in assaying for agents and substances that bind to the described kinases, or have a stimulating or inhibiting effect on the expression and/or activity of these enzymes. In addition, the polypeptide or peptides which are a part of the invention can also be used to assay for agents that, by affecting the association or interaction between the enzymes, modulate their function in vivo. Formats that may be used in such assays are described in detail below, and may comprise determining binding between components of the FAK, ETK or PAK1 pathways in the presence or absence of a test substance and/or determining ability of a test substance to modulate a biological or cellular function or activity in which the activity of one or more of these enzymes is involved plays a role. Assay methods that involve determination of binding between components and the effect of a test substance on such binding need not necessarily utilize full-length, wild-type molecules. For instance, fragments of FAK, ETK or PAK1 that retain the relevant properties described herein may be used. Indeed, as discussed further below, fragments of the polypeptides themselves represent a category of putative modulators, that may be used, e.g. to interfere with interaction between the molecules, to improve it, and so forth. Fusion proteins may also be used in such assays.

Candidate compounds or test compounds include, but are not limited to, those described supra, as well as nucleic acids (e.g., DNA and RNA), peptidomimetics, and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example: DeWitt, et al., 1993, Proc. Natl. Acad. Sci. USA 90:6909; Erb, et al., 1994, Proc. Natl. Acad. Sci. USA 91:11422; Zuckermann, et al., 1994, J. Med. Chem. 37:2678; Cho, et al., 1993, Science 261:1303; Carrell, et al.,1994, Angew. Chem. Into. Ed. Engl. 33:2059; Carrell, et al.,1994, Angew. Chem. Into. Ed. Engl. 33:2061; and Gallop, et al., 1994, J. Med. Chem. 37:1233, each of which is incorporated herein in its entirety by reference.

Compounds may be presented singly or in a library in, e.g., solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull, et al., 1992, Proc. Natl. Acad. Sci. USA 89:1865-1869) or phage (Scott and Smith, 1990, Science 249:386-390; Devlin, 1990, Science 249:404-406; Cwirla, et al., Proc. Natl. Acad. Sci. USA 87:6378-6382; and Felici, 1991, J. Mol. Biol. 222:301-310), each of which is incorporated herein in its entirety by reference.

The use of peptide libraries may be preferred in certain circumstances. The potential for interaction in the PAK1, ETK or FAK pathways to be inhibited by means of peptide fragments peptides and/or has been mentioned already. Such peptide fragments may consist of for example 10-40 amino acids, e.g. about 10, about 20, about 30 or about 40 amino acids, or about 10-20, 20-30 or 30-40 amino acids. These may be synthesized recombinantly, chemically or synthetically using available techniques.

In any assay method according to the invention, the amount of test substance or compound which may be added to an assay of the invention will normally be determined by trial and error depending upon the type of compound used. Even a molecule which has a weak effect may be a useful lead compound for further investigation and development.

In one embodiment, agents that interact with, such as by binding to, one of the kinase molecules described herein are identified in a cell-based assay system. In accordance with this embodiment, cells expressing one of these molecules, or a fragment of these or molecules such as a fusion protein, which contain all or part of the molecule are contacted with a candidate compound AG879 and the ability of the candidate compound to interact with the molecule or molecules is determined. If desired, this assay may be used to screen a plurality (e.g., a library) of candidate compounds. The cell, for example, can be of prokaryotic origin (e.g., E. coli) or eukaryotic origin (e.g., yeast or mammalian). Further, the cells can express a kinase molecule, such as FAK, ETK or PAK1, a fragment of one of these molecules fusion protein endogenously or be genetically engineered to express one or more of these molecules. In certain instances, the molecule, fusion protein or peptide or the candidate compound is labeled, for example with a radioactive (such as 32P, 35S, 131I or 90Yt) or a fluorescent label(such as fluorescein isothiocyanate, rhodamine, phycoerythrin, phycocyanin, allophycocyanin, o-phthaldehyde or fluorescamine) to enable detection of an interaction between the molecule and a candidate compound. The ability of the candidate compound to interact directly or indirectly with the molecule, a fragment of the molecule or a fusion protein can be determined by methods known to those of skill in the art. For example, the interaction between a candidate compound and the molecule, a fragment, or fusion protein can be determined by flow cytometry, a scintillation assay, immunoprecipitation or Western blot analysis, ELISA, IHC, RIA, or any of the other, well known formats for immunoassays.

In another embodiment, agents that interact with the molecule, such as by binding or, an a functionally active fragment, or an fusion protein, are identified in a cell-free assay system. In accordance with this embodiment, a native or recombinant molecule or fragment thereof, or a fusion protein or fragment thereof, is contacted with a candidate compound or a control compound and the ability of the candidate compound to interact with the molecule, or fragment fusion protein is determined. If desired, this assay may be used to screen a plurality (e.g., a library) of candidate compounds. Preferably, the molecule, fragment or fusion protein is first immobilized, by, for example, contacting said molecule, fragment or fusion protein with an immobilized antibody which specifically recognizes and binds it, or by contacting a purified preparation of said molecule, fragment or fusion protein with a surface designed to bind proteins. The molecule, or fragment or fusion protein may be partially or completely purified (e.g., partially or completely free of other polypeptides) or be part of a cell lysate. Further, the molecule, fragment or a fusion protein may comprise the kinase or a biologically active portion thereof, and a domain such as glutathionine-S-transferase. Alternatively, the molecule, fragment or fusion protein can be biotinylated using techniques well known to those of skill in the art (e.g., biotinylation kit, Pierce Chemicals; Rockford, Ill.). The ability of the candidate compound to interact with the molecule, fragment or fusion protein can be can be determined by methods known to those of skill in the art.

In another embodiment, a cell-based assay system is used to identify agents that bind to or modulate the activity of a molecule, or a biologically active portion thereof, which is responsible for the production or degradation of a molecule involved in the kinase pathways described herein or is responsible for the post-translational modification of the molecules. In a primary screen, a plurality (e.g., a library) of compounds are contacted with cells that naturally or recombinantly express: (i) the relevant molecule, an isoform of the molecule or molecules, a fusion protein, or a biologically active fragment of any of the foregoing; and (ii) a protein that is responsible for processing of the target molecule, in order to identify compounds that modulate the production, degradation, or post-translational modification thereof. If desired, compounds identified in the primary screen can then be assayed in a secondary screen against cells naturally or recombinantly expressing the specific molecule of interest. The ability of the candidate compound to modulate the production, degradation or post-translational modification of the molecule can be determined by methods known to those of skill in the art, including without limitation, flow cytometry, a scintillation assay, immunoprecipitation and Western blot analysis, ELISA, IHC, RIA, or any of the other well known formats for immunoassays.

In another embodiment, agents that competitively interact with (i.e., bind to) a polypeptide involved in the kinase pathways are identified in a competitive binding assay. In accordance with this embodiment, cells expressing the polypeptide, fragment, or fusion protein are contacted with a candidate compound and a compound known to interact with the molecule, such as AG879; The ability of the candidate compound to competitively interact with said polypeptide, fragment, or fusion protein is then determined. Alternatively, agents that competitively interact with (i.e., bind to) the polypeptide, fragment, or fusion protein are identified in a cell free system by contacting the polypeptide, fragment or fusion protein with a candidate compound and a compound known to interact with said polypeptide, fragment or fusion protein, such as AG879. As stated supra, the ability of the candidate compound to interact with the polypeptide, fragment or fusion protein can be determined by methods known to those of skill in the art. These assays, whether cell-based or cell-free, can be used to screen a plurality (e.g., a library) of candidate compounds.

In a preferred embodiment agents that competitively interact with a polypeptide are identified in a cell-free assay system by contacting a polypeptide in the kinase pathways a fragment or fusion protein with a candidate compound in the presence or absence of AG879.

In another embodiment, agents that modulate (i.e., upregulate or downregulate) the expression of molecules involved in the kinase pathways are identified by contacting cells (e.g., cells of prokaryotic origin or eukaryotic origin) expressing a polypeptide or polypeptides with a candidate compound or a control compound (e.g., phosphate buffered saline (PBS)) and determining the expression of the polypeptide, or mRNA encoding the polypeptide. The level of expression of a selected polypeptide or mRNA encoding the polypeptide, in the presence of the candidate compound is compared to the level of expression of the polypeptide or mRNA encoding the polypeptide in the absence of the candidate compound (e.g., in the presence of a control compound). The candidate compound can then be identified as a modulator of the expression of the polypeptide based on this comparison. For example, when expression of one of the molecules, e.g., ETK, FAK or PAK1 is significantly greater in the presence of the candidate compound than in its absence, the candidate compound is identified as a stimulator of expression of that kinase. Alternatively, when expression of the kinase is significantly less in the presence of the candidate compound than in its absence, the candidate compound is identified as an inhibitor of the expression of the kinase. The level of expression of the kinase or the mRNA that encodes it can be determined by methods known to those of skill in the art. For example, mRNA expression can be assessed by Northern blot analysis or RT-PCR, and protein levels can be assessed by Western blot analysis, or by the other assay formats referred to supra.

In another embodiment, agents that modulate the activity of the polypeptide or polypeptides are identified by contacting a preparation containing a polypeptide, or cells (e.g., prokaryotic or eukaryotic cells) expressing the polypeptide with a test compound or a control compound and determining the ability of the test compound to modulate (e.g., stimulate or inhibit) the activity of said polypeptide. The activity of the polypeptide can be assessed by detecting induction of a cellular signal transduction pathway, detecting catalytic or enzymatic activity of the target on a suitable substrate, detecting catalytic or enzymatic activity of the target on a suitable substrate, detecting the induction of a reporter gene (e.g., a regulatory element that is responsive to the polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate compound can then be identified as a modulator of the activity of the polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of the polypeptide are identified in an animal model. Examples of suitable animals include, but are not limited to, mice, rats, rabbits, monkeys, guinea pigs, dogs and cats. Preferably, the animal used represents a model of diseases such as an autoimmune disease, cancer, a graft abnormality, an anti-angiogenesis model, one related to functional signaling disorders such as hormone or other endocrine disorders, B cell or T cell disorders, etc. In accordance with this embodiment, the test compound or a control compound is administered (e.g., orally, rectally or parenterally such as intraperitoneally or intravenously) to a suitable animal and the effect on the expression, activity or both expression and activity of the polypeptide is determined. Changes in the expression of the polypeptide can be assessed by the methods outlined above.

Further polypeptides such as ETK, FAK and PAK1 can be used as “bait protein” in a two-hybrid assay or a three-hybrid assay to identify other proteins, including natural ligands, that bind to or interact with one of the kinase polypeptides. For example, a polypeptide as described herein may be fused to a DNA binding domain such as that of the yeast transcription factor GAL4. The GAL4 transcription factor includes two functional domains. These domains are the DNA binding domain (GAL4DBD) and the GAL4 transcriptional activation domain (GAL4TAD). By fusing a first polypeptide component of the assay to one of those domains, and a second polypeptide component of the assay to the respective counterpart, a functional GAL4 transcription factor is restored only when the two polypeptides interact. Thus, interaction of these polypeptides may be measured by the use of a reporter gene linked to a GAL4 DNA binding site which is capable of activating transcription of said reporter gene.

This two hybrid assay format is described by Fields and Song, 1989, Nature 340: 245-246, incorporated by reference. It can be used in both mammalian cells and in yeast. Other combinations of DNA binding domain and transcriptional activation domain are available in the art and may be preferred, such as the LexA DNA binding domain and the VP60 transcriptional activation domain.

As those skilled in the art will appreciate, such binding proteins are likely to be involved in the propagation of signals by the kinase polypeptides described herein, including upstream or downstream elements of a signaling pathway involving the polypeptides involved in the pathways described herein.

The precise format of any of the screening or assay methods of the present invention may be varied by those of skill in the art using routine skill and knowledge. The skilled person is well aware of the need to employ appropriate control experiments.

Performance of an assay method according to the present invention may be followed by isolation and/or manufacture and/or use of a compound, substance or molecule which tests positive for ability to modulate the relevant interaction or affect the relevant biological function or activity. Following identification of a suitable agent, it may be investigated further, and may be modified or derivatized to alter one or more properties, without abolishing its ability to modulate the relevant interaction or affect the relevant biological function. For instance, a single chain Fv antibody molecule may be reformatted into a whole antibody comprising antibody constant regions, e.g. an IgG antibody. Any peptidyl molecule may be modified by addition, substitution, insertion or deletion of one or more amino acids, or by joining of an addition moiety or protein domain. An active agent may be subject to molecular modeling in silico and one or more mimetics of the originally identified agent may be created. For example, modifications to the basic AG879 molecule maybe made in accordance with the disclosures of, e.g., U.S. Pat. No. 5,773,476 or 5,457,105, both of which are incorporated by reference, as well as in accordance with basic principles underlying modifications of heterocyclic molecules. For example, it is routine in the art to make salts of heterocyclic molecules, such as nitrate or sulfate salts, to render heterocyclic molecules more soluble and thus more available for molecular interaction with targets. Any such modification of AG879 is encompassed herein, such that the resulting molecule retains the basic properties of AG879, i.e., the ability to interact with the kinases, as discussed herein. It is to be understood that one can identify such derivatives of AG879 by testing the molecule in question, i.e., the “derivative” in an assay together with AG879. Since the properties of AG879 are known, one can determine the properties of the derivative in question in the types of assays that are disclosed herein, together with AG879.

By “derivative: is meant that the compound in question shares the basic heterocyclic structure for AG879, which is:

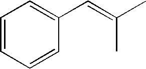

Other compounds are known, such as “LFM-A13”:

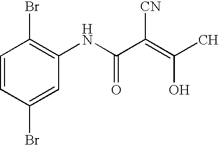

which are expected to function in a manner similar to that of AG879.

The molecules may be formulated in e.g., slow release form, time release form, and in other forms which render them accessible to their target molecules.

Furthermore, an active agent of the invention may be manufactured and/or used in preparation, i.e., manufacture or formulation, of a composition such as a medicament, pharmaceutical composition or drug. These may be administered to individuals, in manners well known to the art.

A compound, whether a peptide, antibody, small molecule or other substance found to have the ability to affect binding between polypeptide chains of a receptor of the invention or binding of such a receptor to a ligand has therapeutic and other potential in a number of contexts. For therapeutic treatment such a compound may be used, alone or in combination with any other active substance.

Generally, such a substance identified according to the present invention and to be subsequently used is provided in an isolated and/or purified form, i.e. substantially pure. This may include being in a composition where it represents at least about 90% active ingredient, more preferably at least about 95%, more preferably at least about 98%. Such a composition may, however, include inert carrier materials or other pharmaceutically and physiologically acceptable excipients. Thus, a composition may consist of the active ingredient obtained using the invention, and an inert carrier. Furthermore, a composition according to the present invention may include in addition to a modulator compound as disclosed, one or more other molecules of therapeutic use.

Also a part of this invention is a method for determining the presence of kinases in a tissue or cell sample comprising contacting said sample with an antibody specific therefor and determining binding there between. Methods for determining the binding of an antibody and its target are well known to those of skill in the art and need not be elaborated herein.

The proteins of this invention may also be used to determine the presence of candidate compounds, such as AG879 or other interactive compounds in a sample by, e.g., labeling said receptor-like binding protein and then contacting said sample with said receptor-like antagonist and determining binding therebetween wherein said binding is indicative of the presence of the molecule, such as AG879. Alternatively, one may determine the presence of AG879 or other equivalent molecules in a sample by treating a cell line that is responsive to the molecule, such as AG879 to two aliquots of said sample, one containing the receptor-like binding protein and one without the receptor-like binding protein, then measuring and comparing the response of said responsive cell to the two aliquots wherein a difference in response to the two aliquots is indicative of the presence of the molecule. In the alternative, cells that are responsive to the molecule can be used in such assays. To elaborate, cells which show some type of response to the molecule, can be used to screen for presence and/or amount of kinases, like ETK, FAK and PAK1 in a sample. For example, assuming that the cell is incubated in the sample in question together with the kinases, any observed change in the response, is indicative of the kinases in said sample.

Other features of the invention will be clear to the artisan and need not be discussed further.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

We claim:

1. A method for determining if a substance is a modifier of a kinase molecule which is also modified by AG879, comprising admixing said substance and said kinase, measuring a property of said kinase, and comparing said property to said property when said kinase is admixed with AG879, to determine if said substance is a modifier of said kinase.

2. The method of claim 1, wherein said property is autophosphorylation.

3. The method of claim 1, wherein said property is cell adhesion.

4. The method of claim 1, wherein said property is phosphorylation of a second kinase by said kinase.

5. The method claim 1, wherein said kinase PAK1.

6. The method o claim 1, wherein said kinase is ETK.

7. The method of claim 1, wherein said kinase is FAK.

8. The method of claim 1, comprising determining said property in vitro.

9. The method of claim 1, comprising determining said property in vivo.

10. The method of claim 1, comprising determining said property on a kinase containing immunoprecipitate.

11. The method of claim 1, wherein said substance in a small molecule.

12. The method of claim 1, wherein said substance is a peptide, an antibody or an antibody fragment.

13. The method of claim 1, wherein said substance is a further kinase, or a portion of a kinase molecule.

14. The method of claim 1, wherein said kinase is a portion of a kinase molecule which has at least one property in common with said kinase, and said property is modified by AG879.

15. The method of claim 14, wherein said portion of a kinase molecule is a portion of a PAK1, ETK or FAK.

16. The method of claim 1, wherein said substance is a nucleic acid molecule or a peptidomimetic.

17. The method of claim 1, further comprising screening a library of substances.

18. The method of claim 17, wherein said library is a peptide library.

19. The method of claim 1, wherein said substance is in solution.

20. The method of claim 1, wherein said substance is immobilized in or on a bead.

21. The method of claim 1, comprising determining said property via flow cytometry, scintillation assay, immunoprecipitation, western blot, ELISA, IHC, or RIA

22. A method for inhibiting a kinase selected from the group consisting of PAK1, FAK, and ETK, comprising contacting said kinase with AG879 or a derivative thereof, in an amount sufficient to inhibit said kinase.

23. The method of claim 22, wherein said kinase is PAK1.

24. The method of claim 22, wherein said kinase is FAK.

25. The method of claim 22, wherein said kinase is ETK.

26. The method of claim 9, wherein said substance is tested in an animal model.

27. The method of claim 26, wherein said animal model is a model for an autoimmune disease, cancer, graft abnormality, anti-angiogenesis, a functioning signal disorder, a B cell disorder, or a T cell disorder.
